(19) United States
(12) Patent Application Publication
(10) Pub. No.: US 2025/0195826 A1
(43) Pub. Date: Jun. 19, 2025

(54) METHOD FOR PRODUCING A CATHETER BODY

(71) Applicant: Heraeus Medevio GmbH & Co. KG, Hanau (DE)

(72) Inventor: Ilias NIKOLAIDIS, Hanau (DE)

(21) Appl. No.: 18/973,961

(22) Filed: Dec. 9, 2024

(30) Foreign Application Priority Data
Dec. 14, 2023 (DE) 102023135110.9

(51) Int. Cl. A61M 25/00; A61B 18/14

(52) U.S. Cl. CPC A61M 25/0012; A61M 25/0045; A61B 18/1492; A61M 2207/10

(57) ABSTRACT

A method for producing a catheter body.

### CROSS-REFERENCE TO RELATED APPLICATIONS

This application claims priority pursuant to 35 U.S.C. 119(a) to German Application No. 102023135110.9, filed Dec. 14, 2023, which application is incorporated herein by reference in its entirety.

### FIELD OF THE INVENTION

The present invention relates to a method for producing a catheter body, comprising the method steps of:
a) providing a hollow cylindrical inner liner extending in a longitudinal direction from a proximal catheter body end to a distal catheter body end of the catheter body;
b) arranging a plurality of electrical conductors on a radially outer surface of the inner liner, wherein the electrical conductors extend axially from a proximal inner liner end region into a distal inner liner end region of the inner liner;
c) coaxially partially sheathing the inner liner and the electrical conductors with an outer covering, so that the electrical conductors are arranged radially between the inner liner and outer covering and so that at least the distal inner liner end region is not sheathed by the outer covering;
d) providing a plurality of hollow cylindrical electrodes having an electrode inner diameter which corresponds at least to an inner liner outer diameter, and a plurality of hollow cylindrical insulating rings having an insulating ring inner diameter which corresponds at least to the inner liner outer diameter; and,
e) electrically connecting one of the electrodes to one of the electrical conductors in each case by coaxially drawing the electrodes onto the distal inner liner end region, wherein, between two axially adjacent electrodes in each case, at least one insulating ring is drawn coaxially onto the distal inner liner end region in order to electric ally insulate the axially adjacent electrodes from one another.

### BACKGROUND OF THE INVENTION

Catheters have been used in medical procedures for many years. Among other things, catheters can be used in medical procedures to examine, diagnose and/or treat tissue while being used in areas of the body, such as blood vessels, that are not easily accessible without more invasive procedures. For example, catheters can be used to deliver electrical energy to selected locations in the human body, such as the heart, to specifically kill tissue, such as heart tissue. This is often referred to as “ablation”. Catheters can also be used to stimulate body tissue. During stimulation, the amount of energy transferred to the body tissue is usually lower than during ablation.

Another procedure, often called “mapping,” uses a catheter with sensing electrodes to monitor various forms of bioelectrical activity in the human body.

Catheters can therefore be used both to transmit (during “ablation” and “stimulation”) and to absorb (during “mapping”) energy to or from body tissue.

Regardless of the direction of energy flow, catheters often contain a plurality of electrodes, i.e., two or more, at a distal catheter body end region, wherein the distal catheter body end region often describes the end that is inserted into the patient. Typically, the catheter contains an equal number of electrodes at its proximal catheter body end region, which are in electrical contact with the electrodes at the distal catheter body end region. The electrodes at the proximal catheter body end region are used to connect electrical devices according to the desired areas of application of the catheter.

The production of such catheter bodies with a large number of electrodes is complex. Typically, the electrodes are electrically connected along the length of the catheter body using wires. These wires take up space that could be used for other things (e.g., guide wires), require a larger diameter of the catheter body, are difficult to handle and time-consuming to assemble. In addition, incorrect connections often occur during assembly and the resulting catheter bodies are not leak-tight.

### SUMMARY OF THE INVENTION

One object of the present invention is to overcome, at least in part, one or more of the disadvantages resulting from the prior art.

It is a further object of the invention to provide a method for producing a catheter body, which is as simple, reliable and cost-effective as possible.

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a method for producing a catheter body, comprising the method steps of:
a) providing a hollow cylindrical inner liner extending in a longitudinal direction from a proximal catheter body end to a distal catheter body end of the catheter body;
b) arranging a plurality of electrical conductors on a radially outer surface of the inner liner, wherein the electrical conductors extend axially from a proximal inner liner end region into a distal inner liner end region of the inner liner;
c) coaxially partially sheathing the inner liner and the electrical conductors with an outer covering, so that the electrical conductors are arranged radially between the inner liner and outer covering and so that at least the distal inner liner end region is not sheathed by the outer covering;
d) providing a plurality of hollow cylindrical electrodes having an electrode inner diameter which corresponds at least to an inner liner outer diameter, and a plurality of hollow cylindrical insulating rings having an insulating ring inner diameter which corresponds at least to the inner liner outer diameter; and,
e) electrically connecting one of the electrodes to one of the electrical conductors in each case by coaxially drawing the electrodes onto the distal inner liner end region, wherein, between two axially adjacent electrodes in each case, at least one insulating ring is drawn coaxially onto the inner liner end region in order to electrically insulate the axially adjacent electrodes from one another.

In a preferred embodiment of the method, the arrangement of the plurality of electrical conductors in method step b) is an arrangement of a flexible printed circuit board, in particular an arrangement of a flexible printed circuit board containing the plurality of electrical conductors, on the radial outer surface of the inner liner. This embodiment is a second embodiment of the invention, which is preferably dependent on the first embodiment of the invention.

In a preferred embodiment of the method, the coaxial partial sheathing in method step c) is a coaxial drawing, or in other words, slipping, of the outer covering onto the inner liner. This embodiment is a third embodiment of the invention, which is preferably dependent upon the first or second embodiment of the invention.

In a preferred embodiment of the method, method step d) further comprises providing a plurality of hollow-cylindrical electrically conductive connecting elements, wherein the connecting elements comprise a connecting element inner diameter which corresponds to at least one inner liner outer diameter, and wherein the electrodes provided have an electrode inner diameter which substantially corresponds to a connecting element outer diameter, so that in method step e) one of the connecting elements is arranged radially between the electrodes and the electrical conductors by coaxial drawing, so that the electrodes are electrically connected to the electrical conductors via the connecting elements. This embodiment is a fourth embodiment of the invention, which preferably depends upon one of the preceding embodiments of the invention.

In a preferred embodiment of the method, the connecting elements comprise an electrically conductive polymer or the connecting elements consist of an electrically conductive polymer. This preferred embodiment is a fifth embodiment of the invention, which is preferably dependent upon the fourth embodiment of the invention.

In a preferred embodiment of the method, the electrodes are ring electrodes. This embodiment is a sixth embodiment of the invention, which is preferably dependent upon one of the preceding embodiments of the invention.

In a preferred embodiment of the method, the connecting elements and insulating rings provided in method step d) comprise lateral surfaces which extend conically in the radial extension. This embodiment is a seventh embodiment of the invention, which is preferably dependent upon the fourth to sixth embodiment of the invention.

In a preferred embodiment of the method, the lateral surfaces of the connecting elements converge conically in the radial direction and the lateral surfaces of the insulating rings converge conically in the radial direction. This embodiment is an eighth embodiment of the invention, which is preferably dependent on the seventh embodiment of the invention.

In a preferred embodiment of the method, the electrodes comprise segment electrodes that are electrically insulated from one another, wherein in method step e) each of the segment electrodes of the electrodes is electrically connected to one of the electrical conductors.

This embodiment is a ninth embodiment of the invention, which preferably depends upon one of the preceding embodiments of the invention.

In a preferred embodiment of the method, the electrodes comprise a coating with an electrically conductive polymer, preferably on a radial outer surface of the electrodes. This embodiment is a tenth embodiment of the invention, which preferably depends upon one of the preceding embodiments of the invention.

In a preferred embodiment of the method, the outer covering and the insulating rings are made of the same material. This embodiment is an eleventh embodiment of the invention, which preferably depends upon one of the preceding embodiments of the invention.

In a preferred embodiment of the method, the outer covering, the electrodes and the insulating rings comprise substantially the same outer diameter. This embodiment is a twelfth embodiment of the invention, which preferably depends upon one of the preceding embodiments of the invention.

In a preferred embodiment of the method, the inner liner is provided on a mounting rod in method step a). This embodiment is a thirteenth embodiment of the invention, which preferably depends upon one of the preceding embodiments of the invention.

In a preferred embodiment of the method, method step e) further comprises a heat treatment. This embodiment is a fourteenth embodiment of the invention, which preferably depends upon one of the preceding embodiments of the invention.

In a preferred embodiment of the method, method steps b) and c) are carried out simultaneously. This embodiment is a fifteenth embodiment of the invention, which preferably depends upon one of the preceding embodiments of the invention.

### DETAILED DESCRIPTION OF THE INVENTION

In addition to the embodiments described herein, the elements of which “contain” or “comprise” a particular feature (e.g. a material), a further embodiment is always contemplated in which the element in question consists solely of the feature, i.e., does not comprise any other components. The word “comprise” or “comprising” is herein used synonymously with the word “contain” or “containing”.

When in an embodiment an element is referred to in the singular, an embodiment is also contemplated in which a plurality of these elements are present. The use of a term for an element in the plural generally also includes an embodiment in which only a single corresponding element is included.

Unless otherwise stated or clearly excluded from the context, it is fundamentally possible and is hereby clearly considered that features of different embodiments can also be provided in the other embodiments described herein. It is also generally contemplated that all features described herein in conjunction with a method are also applicable for the products and devices described herein, and vice versa.

Merely for the sake of conciseness, these considered combinations are not all explicitly listed in all cases. Technical solutions that are known to be equivalent to the features described herein should be included in principle by the scope of the invention.

In the present description, specifications of ranges also contain the values specified as limits. A specification of the type “in the range from X to Y” with respect to a quantity A consequently means that A can take the values X, Y and values between X and Y. One-sidedly limited ranges of the type “up to Y” for a size A accordingly mean as a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastics” comprises a plastics content of >95 to <100% by weight. For example, a “substantially complete filling of a volume B” comprises a filling of >95 to <100% by volume of the total volume of B.

A first subject of the invention relates to a method for producing a catheter body comprising the following method steps:
a) providing a hollow cylindrical inner liner extending in a longitudinal direction from a proximal catheter body end to a distal catheter body end of the catheter body;
b) arranging a plurality of electrical conductors on a radially outer surface of the inner liner, the electrical conductors extending axially from a proximal inner liner end region into a distal inner liner end region of the inner liner;
c) coaxially partially sheathing the inner liner and the electrical conductors with an outer covering, so that the electrical conductors are arranged radially between the inner liner and outer covering and so that at least the distal inner liner end region is not sheathed by the outer covering;
d) providing a plurality of hollow cylindrical electrodes having an electrode inner diameter which corresponds at least to an inner liner outer diameter, and a plurality of hollow cylindrical insulating rings having an insulating ring inner diameter which corresponds at least to the inner liner outer diameter; and,
e) electrically connecting one of the electrodes to one each of the electrical conductors by coaxially drawing the electrodes onto the distal inner liner end region, wherein between each two axially adjacent electrodes at least one insulating ring is drawn coaxially onto the inner liner end region in order to electrically insulate the axially adjacent electrodes from one another.

The method is used to produce a catheter body of a catheter. A catheter body is the elongate, tube-like portion of the catheter which, depending on the application of the catheter, is equipped with a plurality of electrodes, at least at a distal catheter body end, preferably both at the distal and at a proximal catheter body end axially opposite the distal catheter body end. The electrodes at the proximal end of the catheter body are sometimes called connectors.

In a method step a), a hollow cylindrical inner liner is provided. The inner liner extends axially from the proximal catheter body end to the distal catheter body end and radially surrounds at least one lumen extending axially through the catheter. The lumen is used to pass and/or introduce various devices or fluids. For example, the lumen is used to insert a guide wire, which facilitates the insertion of the catheter into the patient.

The inner liner comprises an inner liner inner diameter which is, for example, in a range of 0.75 to 4 mm, and an inner liner outer diameter which is, for example, in a range of 1 to 4.5 mm. Depending on the application, the inner liner can comprise different lengths. For example, the inner liner comprises a length in a range from 30 cm to 200 cm. The inner liner can be made of a variety of different materials, wherein the use of a flexible material or a flexible material combination is preferred. For example, the inner liner is made of a silicone, a polyimide, a polyurethane and/or a polyether block amide, such as PEBAX®.

In a method step b), a plurality of electrical conductors are arranged on a radial outer surface, i.e., the surface of the inner liner radially opposite the at least one lumen. The electrical conductors can be fixed to the inner liner or simply placed on the inner liner. The electrical conductors extend axially from a proximal inner liner end region to a distal inner liner end region of the inner liner. Each end region is the portion of the inner liner which is to be equipped with the electrodes at least distally, preferably proximally and distally. For example, the end regions extend from the respective catheter body ends up to 20 cm towards the axial center of the catheter body. The electrical conductors therefore do not necessarily extend over the entire length of the inner liner from the proximal to the distal catheter body end, but can end or begin before the respective ends in these respective end regions. However, each of the electrical conductors extends at least over the distance between the two end regions, wherein one or more electrical conductors can also extend over the entire length of the inner liner, and thus from the proximal catheter body end to the distal catheter body end. Preferably, the electrical conductors end in a staggered manner at least in the distal inner liner end region, so that the electrodes can be attached to the electrical conductors at an axial distance in the end portion of the catheter body there. In order to facilitate a targeted electrical connection of the electrical conductors to electrodes, the electrical conductors can be or become electrically insulated—except for the portion intended for electrical connection to an electrode.

In a method step c), the inner liner and the electrical conductors are coaxially partially sheathed with an outer covering. The outer covering forms the radially outer layer of the catheter body. After partial sheathing, the following structure results: the inner liner is arranged around the lumen of the catheter body. This is surrounded coaxially by the outer covering, with the electrical conductors arranged radially between the inner liner and the outer covering. The inner liner and the electrical conductors are not covered over their entire length by the outer covering. Rather, at least the distal inner liner end region, preferably the distal and the proximal inner liner end region, together with the respective portions of the electrical conductors arranged there, are not surrounded by the outer covering. At the distal, preferably distal and proximal, inner liner end region, the electrical conductors are exposed and can be electrically connected to electrodes.

The outer covering can be made of a variety of different materials, wherein the use of a flexible material or a flexible material combination is preferred. For example, the outer covering is made of a silicone, a polyimide, a polyurethane and/or a polyether block amide, such as PEBAX®.

In a method step d), a plurality, i.e., two or more, hollow cylindrical, in particular electrically conductive, electrodes are provided with an electrode inner diameter which corresponds at least to an inner liner outer diameter. Furthermore, a plurality of hollow-cylindrical, in particular electrically insulating, insulating rings are provided with an insulating ring inner diameter which corresponds at least to the inner liner outer diameter. The electrode inner diameter and the inner liner outer diameter are selected such that the electrodes and the insulating rings can be arranged coaxially around the inner liner in the region of the distal inner liner end region, preferably in the region of the distal and proximal inner liner end region, and any electrical conductors arranged on the radial outer surface of the inner liner in these regions.

In a method step e), an electrical connection is made between one of the electrodes and one of the electrical conductors in the region of the distal inner liner end region, i.e., the portion which is not covered by the outer covering. For this purpose, the electrodes are coaxially drawn, or in other words “pushed” onto the inner liner end region from the distal inner liner end towards the distal center of the inner liner. The electrodes come into electrical contact with the electrical conductors arranged in the distal inner liner end region, which forms the electrical connection. Preferably, the electrical conductors are electrically insulated except for the portion provided for the electrical connection, so that an electrical conductor can be specifically electrically connected to the intended electrode—without the other electrical conductors which are located in the same portion of the inner liner end region as this electrode being electrically contacted.

In order to electrically insulate the individual electrodes from each other, at least one insulating ring, preferably exactly one insulating ring, is pushed onto the distal inner liner end region between two axially adjacent electrodes. The electrodes and the insulating rings are preferably pushed alternately onto the inner liner.

The insulating rings can be made of a variety of different materials, wherein the use of a flexible material or a flexible material combination is preferred. For example, the insulating rings are made of a silicone, a polyimide, a polyurethane and/or a polyether block amide, such as PEBAX®. Preferably, the outer covering and the insulating rings are made of the same material.

The same steps can preferably also be performed at the proximal inner liner end region to install electrodes there, sometimes called connectors.

The arrangement of the electrical conductors in method step b) can be carried out in different ways. In one embodiment, the electrical conductor wires are made of a conductive material such as silver, gold, platinum or iridium or alloys comprising these materials, and the arrangement consists in laying or fastening the wires on the radial outer surface of the inner liner. In a further embodiment, a conductive ink, a metal paste and/or a conductive adhesive is applied to the radial outer surface, which, possibly after heat treatment and/or irradiation, forms the electrical conductors.

Preferably, the portions of the electrical conductors which are not connected to the electrodes in method step e) are electrically insulated and/or encased by the outer covering.

The electrodes may also consist of an electrically conductive polymer, in particular of an electrically conductive composite material comprising a polymer, or may comprise such a material. For example, the electrodes can consist of a polymer filled with conductive particles. Examples of polymers are silicones, polyimides, polyurethanes and/or polyether block amides, such as PEBAX®. Examples of conductive particles are carbon-containing particles, in particular graphite or carbon nanotubes, or particles comprising or consisting of precious metals, in particular gold, silver and/or iridium.

An embodiment of the method is characterized in that the arrangement of the plurality of electrical conductors in method step b) comprises arranging a flexible printed circuit board on the radial outer surface of the inner liner.

A flexible printed circuit board comprises a plurality of electrical conductors in the form of conductor tracks which are electrically insulated from one another and are preferably arranged between electrically insulating layers. By selectively opening the insulating layers at desired locations, a corresponding conductor track from the plurality of conductor tracks can be specifically electrically contacted with the electrodes in method step e). One advantage of using a flexible printed circuit board is its ease of handling and arrangement on the inner liner, since all electrical conductors can be bundled in a single component. Furthermore, flexible printed circuit boards can be easily adapted to a specific electrode design. Furthermore, the electrical conductors are electrically insulated except for the desired portions.

Furthermore, the electrically insulating material of the flexible printed circuit board can be matched to the material of the inner liner and/or the outer covering, which increases the compatibility of the layers.

Depending on the desired design of the catheter, two or more flexible printed circuit boards can be used, or additional electrical conductors, such as wires, can be used.

The coaxial partial sheathing in method step c) can be carried out in different ways. For example, the outer covering is present as a layer which is wrapped around the inner liner including the electrical conductors in order to achieve partial sheathing.

An embodiment of the method is characterized in that the coaxial partial sheathing in method step c) is a coaxial drawing, in other words “pushing”, of the outer covering onto the inner liner. In this embodiment, the outer covering is in the form of a hollow cylindrical sleeve having an outer covering inner diameter which substantially corresponds to the inner liner outer diameter. The outer covering inner diameter and/or the material of the outer covering must be selected so that the electrical conductors can be arranged between the inner liner and the outer covering.

In this embodiment, the partial sheathing takes place in a process similar to method step e).

In one embodiment, the electrodes are brought into direct electrical contact with the electrical conductors, i.e., without an electrically conductive intermediate element. Preferably, in such an embodiment, the electrodes have an electrode inner diameter which substantially corresponds to the inner liner diameter, wherein the electrode inner diameter is selected such that the electrical conductors can be arranged between the inner liner and the outer covering.

An embodiment of the method is characterized in that method step d) further comprises providing a plurality of hollow cylindrical electrically conductive connecting elements. The connecting elements serve as electrically conductive intermediate elements between the electrical conductors and the electrodes and are arranged radially between the electrical conductor and the respective electrodes to be contacted. For this purpose, the connecting elements have an inner diameter which corresponds at least to an outer diameter of the inner liner, so that they can sheathe the inner liner together with any electrical conductors arranged there. Furthermore, the connecting elements have a connecting element outer diameter which substantially corresponds to the electrode inner diameter, so that in method step e) one of the connecting elements is arranged radially between the electrodes and the electrical conductors by coaxial drawing, such that the electrodes are electrically connected to the electrical conductors via the connecting element. Preferably, each of the electrodes is electrically connected to an electrical conductor via a connecting element.

Preferably, the connecting elements have an axial extension which substantially corresponds to an axial extension of the electrodes. This allows good, reliable electrical contact to the electrode.

The connecting elements can comprise different electrically conductive materials or can consist of different electrically conductive materials. For example, the connecting elements may comprise metals, such as silver, gold, platinum and/or iridium, or may consist of these materials.

An embodiment of the method is characterized in that the connecting elements comprise an electrically conductive polymer. For example, the connecting elements consist of a hollow cylindrical metal ring coated with a conductive polymer. The connecting elements can also consist entirely of an electrically conductive polymer. Preferably, the electrically conductive polymer is an electrically conductive composite material comprising a polymer. For example, the connecting elements can consist of a polymer filled with conductive particles. Examples of polymers are silicones, polyimides, polyurethanes and/or polyether block amides, such as PEBAX®. Examples of conductive particles are carbon-containing particles, in particular graphite or carbon nanotubes, or particles comprising or consisting of precious metals, in particular gold, silver and/or iridium.

An advantage of a connecting element comprising a conductive polymer between the electrode and the electrical conductor is that the connecting element can compensate for manufacturing variations, in particular diameters, due to its certain flexibility. In addition, the connecting elements in conjunction with the insulating rings, which are arranged between the connecting elements, allow the production of a tight, in particular a leak-tight, catheter body. Due to a certain flexibility of both the connecting elements and the insulating rings, these elements can be pushed together tightly, in particular in a leak-tight manner.

The electrodes can have different designs. For example, only a part of the electrodes is designed to be electrically conductive, so that the manufactured catheter body can only be electrically contacted in this direction, at least at this electrode.

An embodiment of the method is characterized in that the electrodes are ring electrodes. Ring electrodes are characterized in that they are electrically conductive in all radial directions, so that the manufactured catheter body, at least at the ring electrode, can be electrically contacted from all radial directions. Ring electrodes thus make the manufactured catheter body electrically contactable, independent of its orientation.

In one embodiment, the insulating rings and the connecting elements have respective lateral surfaces which extend substantially perpendicular to the longitudinal axis of the catheter body. In this embodiment, the insulating rings and the connecting elements thus have a uniform thickness in their radial extension.

An embodiment of the method is characterized in that the connecting elements and insulating rings provided in method step d) have lateral surfaces which extend conically in the radial extension, i.e., outwardly in the finished catheter body. This means that the connecting elements and the insulating rings become “thinner” or “thicker” in the radial extension. Preferably, the connecting elements and the insulating rings have lateral surfaces which extend conically in opposite directions in the radial extension. For example, the lateral surfaces of the connecting elements diverge conically and the lateral surfaces of the insulating rings converge conically in the radial extension. This has the advantage that the connecting elements and the insulating rings can be pushed together more easily and safely in a sealed, in particular leak-tight, manner in order to produce a particularly sealed, in particular leak-tight, catheter body.

An embodiment of the method is characterized in that the lateral surfaces of the connecting elements conically converge in the radial extension and the lateral surfaces of the insulating rings conically diverge in the radial extension. In this embodiment, the connecting elements become “thinner” in the radial extension and the insulating rings become “thicker”.

An embodiment of the method is characterized in that the electrodes comprise segment electrodes, for example two or more, that are electrically insulated from one another, and in method step e) each of the segment electrodes is electrically connected to one, or at least one, of the electrical conductors. In this embodiment, the electrodes, or at least one of the electrodes, are so-called segmented electrodes. Each of these segmented electrodes can thus be used to electrically contact a plurality of electrical conductors, wherein preferably each of the segment electrodes is connected separately to one of the electrical conductors.

The electrodes can comprise different materials or consist of different materials. For example, the electrodes can consist entirely of a metal such as silver, gold, platinum, iridium or alloys with at least one of these metals.

If the electrodes contain electrically insulated segment electrodes, it is preferred that the segment electrodes consist of a metal, such as silver, gold, platinum, iridium or alloys with at least one of these metals, and the insulating portions between the segment electrodes consist of an electrically insulating polymer, such as a silicone, a polyimide, a polyurethane and/or a polyether block amide, such as PEBAX®.

An embodiment of the method is characterized in that the electrodes comprise a coating with an electrically conductive polymer. This coating can be applied to the inner side facing the inner liner, the radially outer side, or to both sides. If segment electrodes are present, the coating is preferably applied only to the segment electrodes.

The coating may, for example, comprise or consist of a poly-3,4-ethylenedioxythiophene (PEDOT).

The outer covering and the insulating rings can comprise different materials or consist of different materials. Suitable materials include silicones, polyimides, polyurethanes and/or polyether block amides, such as PEBAX®.

One embodiment of the method is characterized in that the outer covering and the insulating rings are made of the same material. The compatibility of the materials of the two components improves the properties and simplifies the production of the catheter body.

The outer diameters of the outer covering, the electrodes and the insulating rings can have different values.

An embodiment of the method is characterized in that the outer covering, the electrodes and the insulating rings have substantially the same outer diameter. This allows the production of a catheter body with a substantially uniform outer diameter. The catheter is therefore substantially “smooth”, which makes it easier to insert into a patient.

For example, the outer covering, the electrodes and the insulating rings can have an outer diameter which is in a range from 1.25 mm to 5 mm.

An embodiment of the method is characterized in that the inner liner is provided on a mounting rod (often referred to as a “mandrel”) in method step a). The mounting rod facilitates the execution of the following method steps by mechanically stabilizing the inner liner. Preferably, an outer diameter of the mounting rod substantially corresponds to the inner liner inner diameter. Preferably, the inner liner is drawn onto the mounting rod for the provision of the inner liner.

An embodiment of the method is characterized in that method step e) further comprises a heat treatment. The heat treatment serves to at least partially melt or at least partially soften the insulating rings, the inner liner and/or the outer covering, so that a good, in particular tight, preferably leak-tight, outer sleeve of the catheter body is created. Preferably, the heat treatment creates an integral bond between the inner liner and the outer covering, which improves the structural stability of the catheter body. The necessary temperature for the heat treatment depends on the materials used and is, for example, in a range between 180° C. and 250° C.

The method steps of the method can, if technically possible, be carried out in a different order.

An embodiment of the method is characterized in that method steps b) and c) are carried out simultaneously. For example, the electrical conductors can be arranged on an inner side of the outer covering, i.e., the side of the outer covering facing the inner liner after drawing, so that in method step c) the electrical conductors are arranged around the inner liner together with the outer covering.

### BRIEF DESCRIPTION OF THE DRAWINGS

The invention is further illustrated by way of example below by means of figures. The invention is not limited to the figures.

The following are shown:

FIG. 1 is an exemplary flow chart of a method for producing a catheter body;

FIG. 2 shows exemplary schematic components for use in a method for producing a catheter body, in a perspective side view;

FIG. 3 shows exemplary method steps of the method using the components shown in FIG. 2; and,

FIG. 4 shows a method step of a further exemplary method for producing a catheter body.

### DETAILED DESCRIPTION OF THE INVENTION

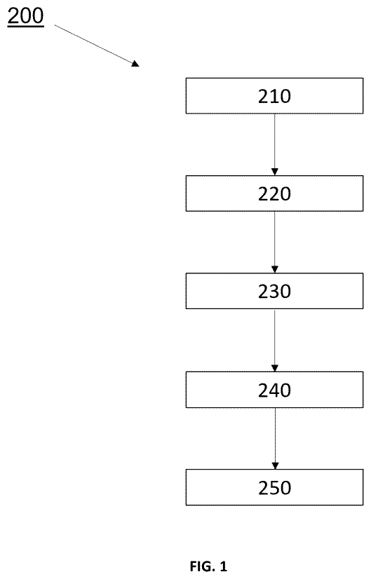

FIG. 1 shows a flow chart of an exemplary method for producing a catheter body.

In a method step 210, a hollow cylindrical inner liner is provided which extends in a longitudinal direction from a proximal catheter body end to a distal catheter body end of the catheter body.

In a method step 220, a plurality of electrical conductors are arranged on a radial outer surface of the inner liner, wherein the electrical conductors extend axially from a proximal inner liner end region to the distal inner liner end region of the inner liner.

In a method step 230 the inner liner and the electrical conductors are partially sheathed with an outer covering, so that the electrical conductors are arranged radially between the inner liner and outer covering and so that at least the distal inner liner end region is free from sheathing with the outer covering.

In a method step 240, a plurality of hollow cylindrical electrodes with an electrode inner diameter that corresponds at least to an inner liner outer diameter and a plurality of hollow cylindrical insulating rings with an insulating ring inner diameter that corresponds at least to the inner liner outer diameter are provided.

In a method step 250, an electrical connection of one of the electrodes to one of the electrical conductors is carried out by coaxially drawing the electrodes onto the distal inner liner end region, wherein at least one insulating ring is drawn coaxially onto the inner liner end region between two axially adjacent electrodes in order to electrically insulate the axially adjacent electrodes from one another.

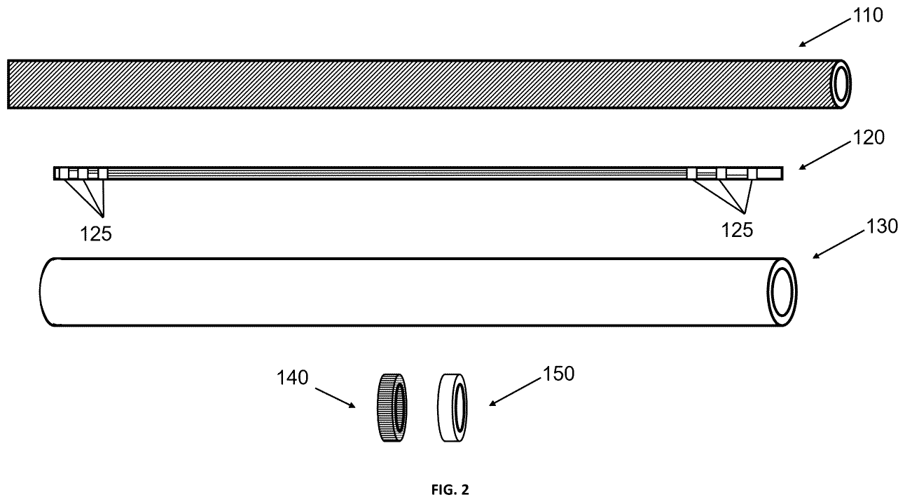

FIG. 2 shows various exemplary components of a method for producing a catheter body in a perspective side view. Shown is a hollow cylindrical inner liner 110, a plurality of electrical conductors 120 in the form of a flexible printed circuit board, a hollow cylindrical outer covering 130, an electrode 140 in the form of a metallic ring electrode (other electrodes are not shown for better clarity) and an insulating ring 150 made of an electrically insulating polymer (other insulating rings are not shown for better clarity).

The electrical conductors 120 in the form of a flexible printed circuit board have contact points 125 at both axial ends for a total of six electrodes 140.

An outer diameter of the inner liner 110 is selected such that it substantially corresponds to an inner diameter of the outer covering 130, the electrodes 140 and the insulating rings 150, so that a coaxial drawing of these onto the inner liner 110 is possible, even with electrical conductors 120 arranged in between.

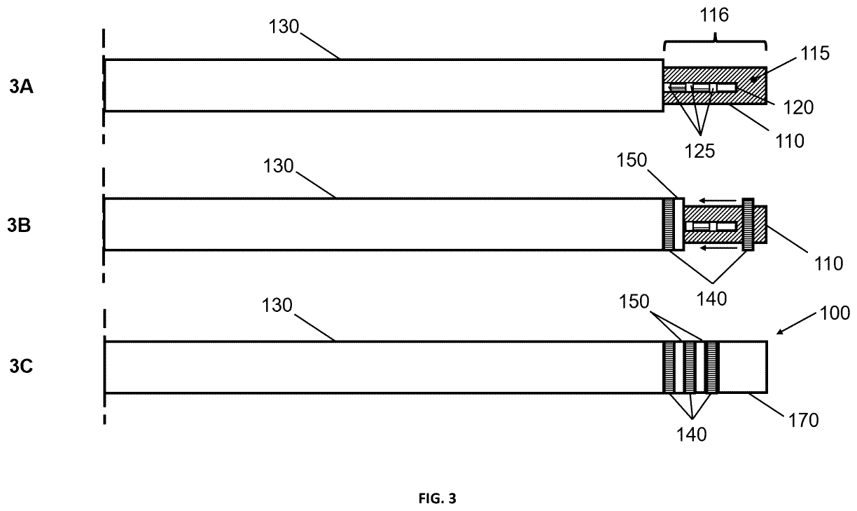

FIG. 3 shows exemplary method steps of the method for producing a catheter body using the components shown in FIG. 2. For the sake of clarity, the method is shown below only for one of the ends of the catheter body, although it is understood that the other end can also be provided with electrodes 140 using the same method steps.

3A shows the inner liner 110 partially sheathed with the outer covering 130, wherein the electrical conductors 120 in the form of the flexible printed circuit board are arranged between the inner liner 110 and the outer covering 130. The electrical conductors 120 are arranged on a radial outer surface 115 of the inner liner 110 and extend axially along the inner liner 110 into a distal inner liner end region 116. The outer covering 130 is arranged around the inner liner 110 in such a way that the distal inner liner end region 116 together with the electrical conductors 120 arranged there, in particular the contact points 125, remain free of the outer covering. The inner liner 110 is thus partially sheathed by the outer covering 130. For this purpose, the outer covering 130 preferably has a smaller axial extension than the inner liner 110.

3B shows the electrical connection of the provided electrodes 140 to the electrical conductors 120, in particular to the contact points 125 of the electrical conductors 120. For this purpose, one of the electrodes 140 has already been coaxially mounted on the inner liner 110, in particular on the distal inner liner end region 116, and electrically connected to one of the contact points 125 (no longer visible). In order to electrically insulate the already mounted electrode 140 from other electrodes 140, one of the insulating rings 150 was coaxially mounted next to the electrodes 140 on the inner liner. A further electrode 140 is currently being drawn onto the inner liner 110 (indicated by arrows), which is electrically insulated by the already arranged and axially adjacent electrode 140 through the insulating ring 150.

3C shows a final catheter body 100 obtained after all electrodes 140 and insulating rings 150 have been attached. To protect the distal end of the catheter body, an electrically insulating closure 170 was attached to the distal end of the inner liner 110 (no longer visible). To ensure stable fixation of the components, the catheter body 100 was subjected to heat treatment.

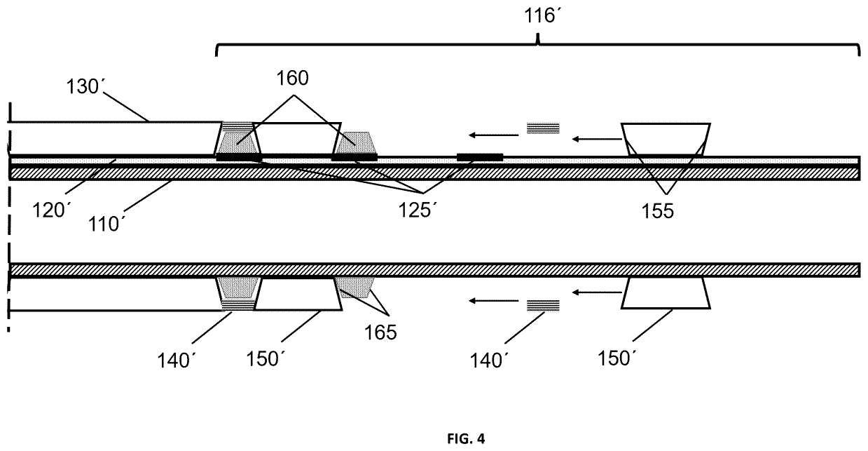

FIG. 4 shows an exemplary process portion of another exemplary method for producing a catheter body. The method according to FIG. 4 largely corresponds to the method steps and components described above and shown in the preceding figures, and therefore reference is made to the above descriptions in order to avoid repetitions. A component which is repeated from the description of the preceding figures has the same reference sign with an apostrophe.

In particular, a drawing of the electrodes 140′ and insulating rings 150′ onto the inner liner 110′ is shown in a schematic longitudinal section. In contrast to FIGS. 2 and 3, in the embodiment of the method shown, an electrically conductive connecting element 160 made of a conductive polymer is arranged between the electrical conductors 120′, in particular their contact points 125′, and the electrodes 140′. As shown, the corresponding connecting element 160 is preferably first drawn onto the inner liner 110′, in particular the distal connecting element end region 116′, and the corresponding electrode 140′ is then drawn onto this. An insulating ring 150′ is arranged, in particular drawn, between axially adjacent electrodes 140′ as shown in FIG. 3. Due to their certain flexibility, the connecting elements 160 improve the electrical connection of the electrodes 140′ to the electrical conductors 120′. In addition, production fluctuations can be more easily compensated.

In the embodiment shown, the connecting elements 160 have lateral surfaces 165 that converge conically in the radial extension. The connecting elements 160 thus become “thinner” outwardly. The insulating rings 150, on the other hand, have lateral surfaces 155 that diverge conically in the radial extension. The insulating rings 150 thus become “thicker” outwardly. The conically opposite lateral surfaces 155, 165 facilitate the production of a tight, in particular leak-tight, catheter body.

The outer covering 130′, the electrodes 140′ and the insulating rings 150′ have substantially the same outer diameter so that a substantially smooth catheter body can be produced.

### REFERENCE SIGNS

100 catheter body
110, 110′ inner liner
115 radial outer surface of the inner liner
116, 116′ distal inner liner end region
120, 120′ electrical conductor
130, 130′ outer covering
140, 140′ electrode
150, 150′ insulating rings
155 lateral surface of an insulating ring
160 connecting element
165 lateral surface of a connecting element
170 termination
200 method
210 provision
220 arrangement
230 partial sheathing
240 provision
250 connection

1. A method for producing a catheter body comprising the method steps of:
a) providing a hollow cylindrical inner liner extending in a longitudinal direction from a proximal catheter body end to a distal catheter body end of the catheter body;
b) arranging a plurality of electrical conductors on a radially outer surface of the inner liner, wherein the electrical conductors extend axially from a proximal inner liner end region into a distal inner liner end region of the inner liner;
c) coaxially partially sheathing the inner liner and the electrical conductors with an outer covering, so that the electrical conductors are arranged radially between the inner liner and outer covering and so that at least the distal inner liner end region is not sheathed by the outer covering;
d) providing a plurality of hollow cylindrical electrodes having an electrode inner diameter which corresponds at least to an inner liner outer diameter, and a plurality of hollow cylindrical insulating rings having an insulating ring inner diameter which corresponds at least to the inner liner outer diameter; and,
e) electrically connecting one of the electrodes to one of the electrical conductors in each case by coaxially drawing the electrodes onto the distal inner liner end region, wherein, between two axially adjacent electrodes in each case, at least one insulating ring is drawn coaxially onto the distal inner liner end region in order to electrically insulate the axially adjacent electrodes from one another.

2. The method according to claim 1, wherein the arrangement of the plurality of electrical conductors in step b) is an arrangement of a flexible printed circuit board on the radial outer surface of the inner liner.

3. The method according to claim 1, wherein the coaxial partial sheathing in method step c) is a coaxial drawing of the outer covering onto the inner liner.

4. The method according to claim 1, wherein method step d) further comprises providing a plurality of hollow cylindrical electrically conductive connecting elements, wherein the connecting elements have a connecting element inner diameter which corresponds at least to an inner liner outer diameter, and wherein the provided electrodes have an electrode inner diameter which substantially corresponds to a connecting element outer diameter, so that in method step e) one of the connecting elements is arranged radially between the electrodes and the electrical conductors by coaxial drawing, so that the electrodes are electrically connected to the electrical conductors via the connecting elements.

5. The method according to claim 4, wherein the connecting elements comprise an electrically conductive polymer.

6. The method according to claim 1, wherein the electrodes are ring electrodes.

7. The method according to claim 4, wherein the connecting elements and insulating rings provided in method step d) have lateral surfaces which extend conically in the radial extension.

8. The method according to claim 7, wherein the lateral surfaces of the connecting elements converge conically in the radial extension and the lateral surfaces of the insulating rings diverge conically in the radial extension.

9. The method according to claim 1, wherein the electrodes comprise segment electrodes that are electrically insulated from one another, and in method step e) each of the segment electrodes is electrically connected to one of the electrical conductors.

10. The method according to claim 1, wherein the electrodes comprise a coating with an electrically conductive polymer.

11. The method according to claim 1, wherein the outer covering and the insulating rings consist of the same material.

12. The method according to claim 1, wherein the outer covering, the electrodes and the insulating rings have substantially the same outer diameter.

13. The method according to claim 1, wherein the inner liner is provided on a mounting rod in method step a).

14. The method according to claim 1, wherein method step e) further comprises a heat treatment.

15. The method according to claim 1, wherein method steps b) and c) are carried out simultaneously.
